SEQUENCE LISTING (24 sequences)

SEQ ID NO: 1 - protein, 285 aa, Mus musculus
MFLLTPVLVAVVCILVVWVFKNADRNLEKKKEEAQVQPWVDEDLKDSTEDLQVEEDAEEWQEAEESVEHIPFSHTRYPEQEMRMRSQEFYELLNKRRSVRFISSEHVPMEVIENVIKAAGTAPSGAHTEPWTFVVVKDPDMKHKIREIIEEEEEINYMKRMGKRWVTDLKKLRTNWIKEYLDTAPVLILIFKQVHGFAANGKKKVHYYNEISVSIACGLLLAALQNAGLVTVTTTPLNCGPRLRVLLGRPSHEKLLVLLPVGYPSRDATVPDLKRKALDQIMVTV

SEQ ID NO: 2 - DNA, 1541 bp, Mus musculus
gaggtcggacttcagaccatgtttctcctcaccccagtcttggtagcagttgtctgtatcttggttgtatgggtctttaaaaatgccgacaggaacctagagaaaaagaaggaggaggctcaagttcagccctgggtggatgaagacttgaaagacagcacagaagaccttcaagtggaagaagatgctgaggagtggcaagaggcagaggagagtgtggagcacatcccgttctctcacacccgatacccagaacaggaaatgaggatgaggtcccaggaattctatgagctcctcaataagagacgctccgtcaggtttatcagcagcgagcacgtcccaatggaagtcattgaaaatgtcatcaaagcagcaggaacagctccaagtggggcccatacagagccctggacctttgtggtggtgaaggacccagacatgaagcataagatcagagagattatcgaagaggaggaagaaataaattacatgaaaaggatgggaaagcgatgggtcacagacctgaagaaactgagaaccaactggattaaggagtacttggacaccgccccagttctgatcctcattttcaaacaagtccatggttttgctgcgaatggaaagaagaaggtccactactacaacgagatcagtgtgtccatcgcctgtggcctcctgctggctgcactgcagaatgcagggctagtgacggtcactaccactcccctcaactgtggtcctcgtctgagggtgctcctgggccgcccctcacatgagaagctgttggtgctacttcctgtggggtaccccagcagagatgccacagtgcctgacctcaagcggaaagctctggaccagatcatggtgacagtatagccagccaacctaaggagatgctggaatgctgggatgacccccacttctttccccccggggctgctggcacctcttactctctgagtcaggtcttctgaaaccagtgtacagggaaggcccctttccttgtagtgatttttttcatactttctagaactttcagcccagtaagttatttttaatggactgttttagtgaatatatggggcaagaggccaaaaagcaggcataacttaatttctctacaaaattataaatgattttttatttacatataaaagtcataaagaagtcacattagaagatttgctatagttcctaaagctccctatgtaatactttttaatttaaatattgactaatcaagtggcccttctgctctctgctttctggtggcattgagtctaagaatttactactgagatctattctgtcactaagcctcatagggattctgccaatcttgaatgagtgacctttaacataagcaaaatatgacactcacagaaccagagaggcatgtctaacctgccatggatgtagctggcaattatctgaacatgagttgcatggagtgtgccggggctggcttgtgctagcacatggaagatggtggttaaattttttgaaattttacaacaaatttttaaaccatttttactttg

SEQ ID NO: 3 - DNA, 14599 bp, Mus musculus
aggtcggacttcagaccatgtttctcctcaccccagtcttggtagcagttgtctgtatcttggttgtatgggtctttaaaaatgccgacaggaacctagagaaaaagaaggaggaggctcaagttcagccctgggtggatgaagacttgaaagacagcacagaagaccttcaagtggaagaaggtaacaacccctgcggtgcccagggctttgccctgcatgtgtttgcgatggtgcgaactgatgcccaggattcagagagcaagccatgctggcaaaccttcgcttctgttgtgaaaacagaacagtgtatagcaaggatggtcatctcagggcacttgccggtaccagcctctacccagtccgaagtcatggaagctaagatctctcccagcccagatcctctgcaggtcaacaccttctgtctcaggggtgggggtggaggcagagtctctgtacagagtgacagcgagcatggccaggttttgattaggaatgctgaaactgagaacgtgacccgtctagggattgggtactttctcttatgccaggccctgtgccttagagacaggagtggcccatgcccagcaagaaaagggtccggtttagggaatgaggtaaaataaggaaacggacaagccctgtggaactagtataaggtgtttaggaaaggcttgggggtctgctggggcattgggaaagagcaaagctgacccgagggtagaggtgtgagcagcaggggcaagctgtgtcaggaggcgcacaaagccagcacccatgttctaatgcctaggagcgattcctggaaggtgaaagatgtgcagacggagaagtgaggcagagagggctggctgtggctggcatgagaccagtgcttgtcagccttcccaatgctgcaactctttaatacggttcctcacattagagtgatttgcgactgtaaaattactttcgctgatatgtttataagtgtaatcttgttagcgttatgaattgtaatgtaaatatctgatatgcagggtatctggtcccacttgggaagtacttcccttaccacagagaagcactgtccttaaccccgacatctgttctaaggtttgatgagcaatgctttggtgggagggtagtggatgcccaagtcacagtgaagagcatcagattttagatattggggaagatggagggagtggctgagtcactgtcatccatggacaagggcagaaagatccaatttaggggacactgtaagggtgtgctgcatttatcatattcatgatgttaaataaaacaatggtcagaacacatcaacttgaggaggtactcagagattagagaaataatcaccaagtgggtactgtgagagagaagactgaggacagaacttgggagacacctgaagagaagagggacatggtagagagatatgctacagaaagaagatgacatggttgaagagtcttgaggaccacaacacactcacatagcatgcagtggacccagctggtgacacatcatatagagtaagcccttcgtcttcatggtaatacaatgaggtaggcattatcactactatcactactatcactattgcaggagactgaggctgtagtagggtactgcctacccaggcaggtaggactctggcagtctatttccagggtccaagcatagagcttttggctgggtagcctttctcacagaaaagcctagaacagaggaggcttaaaaagagaaattcagaggtagatttcttccctctcaaggccacattcctgggagcagtcaggagttcaagagcagtgagaagtggccctgcttgtttttaggaggttcctgctagctctccactgctgtctctgctgtctgccttttctcaggaaatggtctacccactacccagctcctgaaaccagaaacagagaggacccttttccttctctgccaggaagcaagtcccctgagttttgtcaccatgatgggttgggaatctgttgttccttcggcttccctgatgggccaagttagcatcctccctgtgtaagatatgggcacagcttctgtgtggcttttcttgagcgttcttggttcttgtctctgtctaagatgcctctccataagcatgttagagtcctctgcttacttcccgtgggtggccttctgtgacaccaaaccttagtccacatgtgcacaagaagcacattccatgggcccacacaccctgccttttgggctactccacgttctgtgaaaggcctcccttcccttgtccagtgagactcagccatgatacctttcagctgctcacactggcctgaaacacatcctttccactgtcactcttcccaacagactattaaaaacatgtattataaaattcaatgtttttattcactgaaaacatcattagacaccgaacaaccttcatttctttttacttgttctgtgatatttttggtgtgtttgtgtatgcaggcatgtatatgccatgtcatgcatgtatgtaagtcagaagacaaattgaggagtcggcactctcctctcaccatgagagtccttgggattaagctcagcctgtcaggcttggtggcagtcacctttaccaactgagccatcttgctgtttatctgcctcagatgctgacagctcagattccttcacagcctcagaaccccgactcctgtcccttgttttccttgcctgggtcattcctccaaatctctataacttcattaaaaaatgtatatatatactccattgggatggttacattttattattctatccttaaacatacttattggtttgtttgcatatataagctctaaggatatagaaatcatgcttatttatttataaattcttgataagatcttgttgagcaaattgcctattttaatattccagggtatgggagtattgattaatataacttgtattatacatgcatgatatttcatgttttgcaactacgtaactaagacattggaaaaaattcccaagtcaatcatacccacaaaatgatcttctctcagcccagaatatcatcttccccaaccatgataaagccatggttacaggcatctcccgagcacaagcctggaggagaaaattgtacatccactcactgtctaagacaatcattgtcaggataggacttgctttttcacactttgcttcttatagccagtgtatgtgtagcatattgtataaacaagccaagcttgccgtactggaaaacaaaacagtcagacaacagaacccacaaccaagcaaccaaccaacttctgaccaaacaaacacaaagcaacaacagcaaagttttaaaaacagtctgaatcttaggtactgtggtatgaacaaagcagggtcagataaattcctgaagctcactaaagccaccctagtcaacatggtgtccacgtgtcttagttagggttttactgctgtgaacagacaccatgaccatggcagctcttataaaagacaacatttaattggagctggcttacaggttcagagcttcagtctattatcatcaagacaggagcataacagcatccaggcaggcatggtacagtaagagctgagagttcgacaaattcacctgatgactgctagcagaatattgacttccagggagataggagtagggtattaatggccacgcccacagtggcacacctattccagcagggccacacctcctaatagtgccacccctgagccaaacatacaaaccatcacaccatgccagtaaagaaaacctatttccaaaaaagatgaagggcatccaaagttgttctctgttttacacacacacacacacacacacacacacacacacacggggtggagtgaggtggggagagagcaagagggagagagagagagtcagcttcgcatgctttcaggagactgatgcctcttaatgattcttgagaactttgagagaactaagttcacggacctatgcactctgaagttcagccccatgggctcctgctttcttctgtgatctaacttacttatgcagaacatttacatttaatattccatgtcatccccaggtccccatcactcatacagcaggctaacattcagagtacacccctcccccttctaaagaccttttcagatacactttgtaggcctgccttttggtatcctctgcaaactccatgtccttcatgccctcctccccaccttacggatgccactataactctgtggcttatttctttctttgcttttcttcatggtcctaaataaactggctctgttttttctttatctgtcatctgtattctctgtcttttgctctttactttattttagatttttatttttctgacttgccaacaatttaatctaaataattctagttagattaaaataatctctagagatttttataagtatatcttttttcttctctaagcacagtgtcatagttgcttgctcttctataaatttttagagggctggcacacatgattttatttctaagcattgatttactaactttaatcaagatattctgttctctcgggtgcaggattgcattggtggattactggaaattgtttctaaaattgtttagatattgtcatttgctgctgcttgacaacttgggtgcttccaagaagaaacaccgagttagaagagtaacacaccaaaaatatcctaggctcgtgaaggatgccatctttacagtccatactttcccaaaggatgtaacttgaaggtctgggctgatgcagcttcctattgagtgtgccttgtgggctatggtttggctcgcccttgatgctcaaggattaatgtggagatggacctgtaggagctggcagatgtaagcaagaaatgtgagcctctgggttttggtagcccagtgctttgacaaagaccaaaccaccttatttggtgacctgaatggagactgtgtcaaataacccactctaccatttaacagaatttctgtttgttattttttttttgtaatctcttttgttttgatttgtaataagtctagaatctgaagaatgaccaagatcagtggtcaggattatacagatctacattttaatttttctttttgtaaaaaatttggtagattaatttggctttacatcaccaaatagacttttctggaactttattctcctgaccccaactgaccttataacaagggagatggctgaatttgctgtgctcctggctcttgtttgcagatgctgaggagtggcaagaggcagaggagagtgtggagcacatcccgttctctcacacccgatacccagaacaggaaatgaggatgaggtcccaggaattctatgagctcctcaataagagacgctccgtcaggtttatcagcagcgagcacgtcccaatggaagtcattgaaaatgtcatcaaagcagcaggttggtaactggatgctgagccttgccgaatatcagatgctgtgacagagacagtgttaggacagcactgaagtccaaacatatagacaatgccagagtggtatgtttatctatgaaggcgttgtagttttggctttactgctgtgaagagatatcacgatcaaggcaattcttgtaaggagatttagttaggactgatttacaggttcagaggtttagtcccctattaccaagatgggagcatggcagcttcaagacaggcatggcactggaggagctgagagttctacatcttgttcctaaagaaaacaggagactgtcctcaggcagctaggaggatggtctcaaagtccatgcccacggtgacacacctcctccaacaaggccacacctcttaatagtgccacttcctgggccaagcatattcaaaccaccacaaaggctttcccattgttaatgactgccagaaacacggatccctctttcctacattttgattccccatttcttggaaaacagatgtgatggccattaaaaacttttgaagtcaatacattttatgaattttgtttaaagctaaaaattctgaatgtgttggtatacattgcaggagaaacatcttgtccctcctcaaagcaggctccttcctccacattcagttcagatttatgtttgtcttccaagaaacagactccatgacactctagtacagtaattggctgaatgagcaaaataggaaaatttccttatgtgggtttgtaagttaaactaaagcaagatagatttgctttaaatgtaacctaatatctccacccaggggaaaataaaaacaatgatgctacactctcctctaggattgtcttttctgttgtcttacacctctctaagacacatttacgttcatgttcatgcttgttgcatacccattgagagtcatctttaatccaaaacaggcatcaagagtactttatttcagtcaagaatacaaacaccttggatagttgtatcctttacctaaatggcaacagtcaataatcccgaccctgtgagagtgtgagcttaaatttctcctcctctttctcctcctcttccgtctctttctctgtctgcccttcttcttccttttattgctttttgacacaaggtcttacttcaaataatgatcctcttgcctcagcctcccaactgctgaatgggcttgcagacatacatcagcacaatacctgctactgtatctcttcttaatgaacagttttgcatccctctgccgtcttacacttacttttcatttaatggacaataggaacagctccaagtggggcccatacagagccctggacctttgtggtggtgaaggacccagacatgaagcataagatcagagagattatcgaagaggaggaagaaataaattacatgaaaaggatgggaaagcgatgggtcacagacctgaagaaactgaggtacagagctgatgcaattaggggtgcctggcttgctgttactgtaagaaattaccactgcaggtcctgggtttgtggggtttaaaatgattgttattatcttatcttgatgatgtattaagtgttcatattaatatggctcagtgtcgtatttcagtgcatggatacagaatcagattttaccttgtttctcttttgtttcttcatcttatttttaacccttcgcaaaggctctgacccaaataattggggttttttattatttttattttttttaatatttattttaatttattatactttattataagttgtacattattatttaacttattaaaatatatgtgtaacagagagagagagagagagagagagagagagagagagagagagagagagagagaggaatcagagagaggtggaggtattcacgtgcaggttcttttaggaggcagaggtagcagatctcctggagctggacttacaggcactttgtagctgccttatgtagttgctggaaactgaacttgagtcccatcgaatagcacaaagtgctcttaactgctcagccatctctccagcccagaaactgctgttagttaaaactgaagaattacttcactttccaccgtctggaacataagattgtgacatgatttccaggatcgtgaatgaagcgaatcctggtttgctacagtgacaggatcattccaaaccatgttaccacttaagctttaactaagggcacccttagtaggtctgtactatcaaatagtcattagtgctgtgggctcctcccttccaaagaaaacagagtggaaatcagagtggcctggggaaggggcgggggacaaggtgtggggggggcattggctctcacctctcagaaagccttaaaggaggcgagcccaagttctcagctccagatagtgcagagaggcctgctacttctgtctccaacggtggccttctgtggccagctctttcacctgccatctgtcaccccacagtgacaggggcctgagaaagattgttgtgatcacaagctttgtgataccgtgggacttctagcctaggcttggtcatgagaataaggagtgatgggtctcttggccctgctattattgtgagaggataattttcccttaagatgtttatactatattggaactaatgtatcatggagtgtgttataccacactatgctctcagataagtgtgggatatcagcaatgaaactctaaagcttactgtatattcacaaagaaaatggaaatgtcaaagtcaaggtgggctctgaatttagtgaaacacaggattccagctggacacagtaccttcagaaaccatcagcccctctcttccagtcactgagaatcgttctctcatctatgtgcccattgtccttatgtatgcccttcatatatctaaaatagctaccacactcagcactttaattattagtttgctttttttctcatttcatagacgtttttgtttagaaatctataatacaaatcaagttgtttaaccaacagtgtgtgcatagaaacacacatacacacactcacacagacaagcataagacatattcacatgcacacacacacacacacacattgaatagcaaatggacactatgcccagtcaccttttccagatgtctttgaagcccatatttttaatatataatgtaatgtttaatctatactgtttcaggcttgcatagctatctgtctagatgtgaaaatgccgactctgccctcagtaatcctcagtagtcctgcagtctttttatttgctctaatatgaggtcttaatgaattagtataaaactgaattaactgagactaaacaaaaaaagatcagttgataaagtatgcttagaaaaggtaagcacaaaaatatttatgaatgatcaacttgccttagaagccaggcagactgcttaatcttcctcaaatattttaagaaatttgcattgctaacttcttctctatacatattttatacacgtttaaaatctagttaaagccagagaaaagactaggtggttaagagcacttgctgctcttgcagaggacctgggttcagttcccagcagctaccctacagctcacagtggtctgtaattctggccctaggggatccagtgtcttcttttggcttccctggacaccaggtatgcgcatggtacacatacatatatacatacaagacactcatacacataagacttttaaaaataggtattttaaaagttaattttaaccattagggatatgatcagaatcttaaattatgaaaatgtccaaaatataatacaatttaaatagtgaaatgtagccttggggcctgggcagagccttttggcagcattaagcttcctcaatgaatgatggaaaccagctgctgggcctgcttgtggcctgaaggcagcatgtgttactggcaaccgttccatgctagaaatctagtgagtccaggaggcgatcaaaagggatctttttatctgtcatttctgtgatctcaaagtgtgatgtatgcatcagtaaataccactttccataccttaacttactccaccctcaggagggccatgcccaggaaccagctactctggcttcagagctgacctcaatcattaatgttccctctctgtgagggggaaaacctactcaccagcgggactaagctggttaaggtgagagggcagatccccaaaagcttgacatcagcaggatatacatttctgttgtgttttaaaatggtgactggtaaccaggctaaactgtaaaatgaatagtcaccatttcttgataattatatgtaatatcccatgggggaaaggcagccgtttggaatggatgtttatacaggcccgtgtctctcttctcagcacagaatagtttcagcacagatgtaggaggcagggtagtggagtgaaggaggatacgtttagagatggaaaacagaaatttcaagtcatcattactatccacatgaatggccactggctcccctcagacgcgccctgcctctttccacagctaggatttcttcatctgctctttctcattgccaccattgctgtccccttcacttgcttcttcccttgttctagcaaatgtctgtagtctttacctcttccctcagttctccctggcctggccctcctttgaccaccctcctgcctgggacacagccatagtccagtgtctaggttccctcccactatgatgactgtgtagttggtacctcacagccactcttggtgatgacattctcatcagcacagcccatgcttctgaaatggacctgatcacatgatgctgctgctcagcccaaggtaaaccctccatcacttaccgtattagggccaggaccctgcaaaatctgtgtgtaccttccagcccacccatgtaccggcatgccctccatcctgatctgccatacatcaacttttctgattggctacactcacccattctttcttccagggcgccactccctaactcccctccctcatcttcgttgcccaactggaggccattttctggtttccaatggtattttttcaacgttctttttcaaaaccaacactttgctttcagcgtggtatttgttatctaatgcattaggagcattcagcttcattttggcgaacaccagcttggtgatctaaggctattgggtctttctgttgcaggacatttgatcacactgtgaaccctgagattgtcctatttactggggggaaatctgtttctagttgtggtgtggcccagccttagcacacacctttaatccctctggctggaatacagacatgcccttagtacacacctgtaatcccaaacaatgaaggtaaagttagtttatagaaagatacacctatgtctgaaagtgatgtttaatcgagtggcaggcaaagcagcaaatcagagaaagacttgacagaataggatatttccagctctccagagaacaacgagggaagagaggtgacttaagagagtcagagaaagaggggagaaggcagttttactggaacagttgtacagagacaggttgcagagagaacaagccagacacaggcgaagacatcatgagccatagaagcagaaggagccagaagattacaacagattgcctaagttagtctgaggtcaagcagagcaattcaggagaagccaagtagaaccagtatggagaagaatttgggccagagcagctgaggtgaaccagccagtcagagaccagaaagaacaagaaagggtgagcttattcagcagtaagtctcagaggctgaaaatattctaggcctagataagattgtacagaggctagaaccttccatgactaggcccggtcagcagatagaagcagtaagtaactggagacaattaatacaggagaataaaagatactcttacatctttctgtctccttcagcattttgcctgcttttccatgatgggcaagcactaatgtagatttatcaatttcattaaccttatataaaaagataaatttgacaaaaaataaaaaatctagtctttgtcaacttcaatagaaaaggacaacaaaatgtgtatttaaaaatcatagattagctaaggaagtttttaaaagggaaagggaaaaaaaaaacctaaaaattcagagtaaatattctcctgaatgttctttctactctgtccccaaagaaccaactggattaaggagtacttggacaccgccccagttctgatcctcattttcaaacaagtccatggttttgctgcgaatggaaagaagaaggtccactactacaacgagatcagtgtgtccatcgcctgtggcctcctgctggctgcactgcaggtgcgctgcacacccagaggctcaagggggacaagggagacaggggaccaagaatgactgtagtggaaagtttcagccaagtttccaactgggggtcattggggaattgtttgcctagagctgagttcctcagaaagataagtccgatgtgacaacaacccctggaaacctggattggtgggtaacatatttctgcaagctgaaattctccctggaaagtcacttgtaatagctcagggtgacaagaactgctgtgattgctttcttccataatagcatgtagcttgagggtttgagcagccatctgagaagaaaaccaaagcctgaaagatgcactcccctcatgtgtactccactcctttccagactggtgggatgattaaacagctaccaggctcttatcggccttcctgagatgcttacaagactgtgttgtctcagatgactgtgtgcttacaaactttcctattgagatgcttgagaaacgtaaatgtgagatggagcagggctgagttctgcctcacaaaccctgtgccatgtctgggacacactggggttagttaacttataaacagtttatctgatggacacttgggattcaggtcaggagtgagggagtgatgctaaatgtacaaattaacgcaacaaacaatattgtaagagagaatggccctgcttgaagaaagggtcaattctctctctctctctctctctctctctctctctctctctctctctctctctctctctctctctctctctgtgtgtgtgtgtgtgtgtgtgtgtgtgtgtatgagcatgtgtgatgagattcctcgctgtctcaaagaaagatgaggcttgtatctagcggcattcatatcgctactgctgcttctcaaaccatcctgaggaagctggcttttgctttccctcctcacaatgtaggcagataaatgaaggcgaactagtgaccagagacagccagagtcagaccagatggcactgcagatgagatagagtttaggggaagcagaagaagccagcaaccttcccacaatgctcttggcctttgaaaagggctggccaggctagcttttctgttgagcttgctgcctctgctttctcagaattccaaggtaggttatgactgaggtgtgagaaaaactcttcctggagcctgatcaccagcatagtgactcattgggctgggctggccatgcccaaagctcctggccttcctcctggctcaggctccaaggtgggcaggtgactggggaaacatgggttggaggggaaacagctagggacctagagggcactctctgtctgtatagatgcatcttgtctgcttcccccacaagtgtcgccatgcctcgatttgtattgctttaatatgagttctgggaggttgggctcatccttatgcttaccaggcatgtaccttactgatggcatgatctccctaggtattgtatgatttttatggatgatgatgtgtgtaagtcctcaggagagcctgcagtaacccactccagatccctataattatatgagttcaaatgttgctattatggctattaataactaaatatagccagacaggtaactggcattctggaaaaaaacatgtcaggaaaattctagtggacttcatactgatggactgctggggctgttgtgtcagataaaaggtacagactggccactaaagctaaacacttatcttcgtcattctttaataatggtttcactcctcaaccttgtttctctaaagcagccatttccattgttcaagcatcattggaaagtaagatgagttattcttccagatgtttccagttgtttgtctaaatgaaggcaacaggttgaggggaacaggcaaggtttacaacacatcacctgggctggaagcttctttgctgcctgacttcaggggaaaatgaaatggccccaggctgggagaagagctgtggcttcgcctgattttcaaatgctcatttgttctctcttgtttcagaatgcagggctagtgacggtcactaccactcccctcaactgtggtcctcgtctgagggtgctcctgggccgcccctcacatgagaagctgttggtgctacttcctgtggggtaccccagcagagatgccacagtgcctgacctcaagcggaaagctctggaccagatcatggtgacagtatagccagccaacctaaggagatgctggaatgctgggatgacccccacttctttcctcccggggctgctggcacctcttactctctgagtcaggtcttctgaaaccagtgtacagggaaggccactttccttgtagtgatttttttcatactttctagaactttcagtccagtaagttatttttaatggactgttttagtgaatatatggggcaagaggccaaaaagcaggcataacttaatttctctacaaaattataaatgattttttatttacatataaaagtcataaagaagtcacattagaagatttgctatagttcctaaagctacctatgtaatactttttaatttaaatattgactaatcaagtggtccttctgctctctgctttctggtggcattgagtctaagaatttactactgagatctattctgtcactaagcctcatagggattctgccaatcttgaatgagtgacctttaacataagcaaaatatgacactcacagaaccagagaggcatgtctaacctgccatggatgtagctggcaattatctgaacatgagttgcatggagtgtgccggagctggcttgtgctagcacatggaagatggtggttaaatattctgaaattatacaacaaattattaaaccatttgtactttg

SEQ ID NO: 4 - protein, 453 aa, Mus musculus
MSNISSIVNRARDAFNSGKTRPLQFRVEQLEALQRMINENLKGISKALASNLRKNEWTSYYEEVAHVLDEIDFTIKGLSDWAEDEPVAKTRQTQEDDLYIHSEPLGVVLVIGAWNYPFNLTIQPMVGAIAAGNAVVLKPSEVSDHMADLLSTLIPQYMDKDLYPVIKGGVPETTELLKEKFDHIMYTGSTAVGKIVMAAAAKHLTPVTLELGGKSPCYVDKDCDLDVACRRIAWGKFMNSGQTCVAPDYILCDPSIQNEIVEKLKKSLKDFYGEDAKQSHDYGRIINDRHFQRVINLIDSKKVAHGGTWDQPSRYIAPTILVDVDPQSPVMQEEIFGPVMPIVCVRSLDEAIKFINQREKPLALYVFSNNDKVIKKMIAETSSGGVTANDVIVHITVPTLPFGGVGNSGMGAYHGKKSFETFSHRRSCLVRSLRNEEANKARYPPSPAKMPRH

SEQ ID NO: 5 - DNA, 1722 bp, Mus musculus
ccaggagttccagctgctggagaggactgtgtagaaggtaacctaccccatctctcttacttcgtctctagatgggagcagacaagtacatatagcctgcttggagcgaggactttgaaaggctgagacttgcgtccactctgagggcaactagtccagactctgacagttaccatgagcaatatcagtagcatcgtgaaccgggcccgtgatgcctttaactcaggcaagactcgaccgctgcagttccgggttgagcagctggaggcgttgcagcgcatgatcaacgagaacttgaaaggcatctccaaggcgctggcctctaacctgcgcaagaatgaatggacctcctactacgaggaggtggctcacgtgctggacgaaatcgatttcacgattaaggggctctccgattgggctgaggacgagcctgtcggaaagactcgtcagacccaggaagatgacctctacatccactcggagcccctgggcgtggtccttgtcataggtgcttggaactacccattcaacctcaccattcagcccatggtgggtgccatcgctgcaggaaatgctgtggtcctcaagccctcggaagtgagtgatcacatggcagacctgctttcaacacttatccctcagtatatggacaaggatctgtacccagtgatcaaagggggtgtccctgaaaccacggagctgctcaaagagaagtttgaccacatcatgtacacagggagcacggctgtaggaaagatcgtcatggcggctgccgccaagcatctgacccctgtcaccttggagcttggaggaaagagcccttgttacgtagacaaggactgtgatctagatgtggcttgccggcgtattgcttggggaaagtttatgaacagtggccagacctgtgtggccccagactacatcctctgtgacccctccattcagaatgaaattgtggagaagctcaaaaagtcactgaaagatttctatggggaagatgctaagcagtcccatgactatgggaggatcatcaacgaccggcacttccagcgggtcataaatctgattgacagcaagaaagtagcccatggaggcacctgggatcagccttcacgatacatagctcccaccatcctagtggatgtggacccccagtccccggtgatgcaggaggagatctttgggccggtgatgcccattgtgtgtgttcgaagcttggatgaggccattaaatttatcaaccagcgcgagaagcccctggcactctatgtgttctccaacaatgacaaggtgatcaagaaaatgatcgcagagacatcaagcggtggagtgacagccaatgacgtcattgttcacatcactgtgcccactttgccctttgggggtgtggggaacagcggcatgggggcctaccatggcaagaaaagcttcgagaccttttcccaccgccgctcttgcctggtgaggtctctgcggaatgaagaagctaacaaggccaggtatcccccaagcccagccaagatgccccggcattgagagggctacttcacctgacacttgttttgctggctgtcctgtccttgaagaattgctcacggagcctcatcctggcttctctcccacctgctgccccatgcatgctcaactacccgaaggctctcacctcccccctcttgttctgctgtttgctgagcacagagaccaataaaaaagcctctaaag

SEQ ID NO: 6 - DNA, 9670 bp, Mus musculus
ccaggagttccagctgctggagaggactgtgtagaaggtaacctaccccatctctcttacttcgtctctagatgggagcagacaagtacatatagcctgcttggagcgaggactttgaaaggctgagacttgcgtccactctgagggcaactagtccagactctgacaggtcagcatttcctgactgggtgcactgaatgccaagcaccagaggtctgtcaccttccgacattggaccaagagagtcccagagaccctcaaagacacaggaacagaggtgtcctttgggtgagagacctgtgccctgccaagcctctcagcccctagaaaggcggcagggctgagtagctgagtgtgtgcaacttgggagcagcctgatttcagtgcttgtcacctgtgagggcaaaggcagcatgcttaatgccatcagtcctactcttctcacccgtggaacagacgcataactgaccttttttcgtgaccactattagggtgcatttaaaaatcaatctctctcttttctgctcctctttctccaccttccctcatgtgtgtgtctgtgtgtgtgtgtttctgtgtgtgtgtgcgtgcaccatacaggtatatgtgccatgacatgtgtgcaggtcagtagacagccttggttgtcagtccctgccttctactttgttcaaggcagggtctattctttattattgactgctatccaggtacaccaagacagttggcctgtgaggttccagggagtctcctgtctctggcccccatcttatagcaggagttgtgagatttcagaaatgtgcacccacatctagctttatttggcatcttggcacccaaacttgggttctcatgctttccaagcaaggacttcacccactgaaccatctcaccagctccactatggcggttttctgaaactgaagggagtggggagaaggcgctgagtgtgaacgggtctggaaggcgggtataacctttaaggctggctggttctgagagaggaaagccggcttgtgtcccattcaggtgccaggtgcagcatcaaatgtggctccacccaacggtcagtaactccgcagacagcaccgtggttaactgcctcacagagggggcccagggacctagttccttaaagcccacttagttttgagagacgacatggaggggcaagcccagccctgtccagctgcattcacacgaggcccttctcctccgaggaccctacctttgtgtttagtggctgaggctgtgtggctctgcttgaagctctggctatcaggaaggacctggcccaccggctggcaggacaaactggcccagtgaaggcactgtccggtctcctggtggatcacaaggaaaggggcgtggcatgtccagatttgaaccctggatgcctcttcccagggtaggaagtctaagggtcaacatgaataggtgagggtggtggtggaaagaatctgcatgcaaaatcaggcactcatgtctgacatcgtcaaacactatatttatgtagatttatatgggggtgaggcttggggagcacttgtatttttgtgtgcaattgcaagagcttcctccacattgcagtgttaaatatcatatggtgctggaccaaccatctctggaaggagggtgagggtttggtacccgtgactatggttctctgtgtagtagaaacggcttcttgggaagagagaggaagtgctttgaaatgcatcctctcttgtttcattctgcatcatggccaggtttctctgcacaaagatgcaatagacattcaggaaaataagcgcattgcaagatgtcaaaagtcatgaaaaatgaaaagcaaggtcactgcagctggggtgtagtgtggctgtagaagcacgtttatgatgtacaggttccgggagagggaggaagagagagggagggaaggaggaatggaggaagagagggagagggagcaagagaggggggagggagggaggggagaagggagagagagagagaagaaagagagggagggaagaaggaatggaggaagagagagaaagaggggaagaaagagagggagggaaggaggggtggaggaagagagaggtgtgagggatggagggacagagagggagggaaggagaaatggaggaagagagaaacagagagatggaggaagagagaaaagtcaccaattatttttctccccagtcgccccctgccctcacaccaaggctacccccacttttctttcactcatgcacacacactacacccaatgtttaggtaacacatgtagggcaggcagagcctttgtgaatgtgcttggtagttttctgccttacttctaaataaccaatcacacggatgccacagggttcctttagactgtccagggctccggggctcttagactgttcccaatgtgggtgtcacatgcaaacagtaagcaccttcttgtttgtggtgcccaggatgctgcaggtactagaaattcctgcacagacctttgctacttctacacagacttaccctttaaaacatttaattatatgtttttttcagcataaagatctattttgattatttttagttgcgtgtatgggtgtgtgcctgtgtgtgattgtgtaattgtctgtatgggagtgtaagtgcctacagaggcatcggatcccctggagctgccgttacaagtaagggactgggactcagacctgagtggtttggatgagcagtgaatgagcgtggtgagccatcccaccatgccctataaacactttcaagaggactttgctgggtatggtcaagttgtccccctgcgacagccaccccaacttgccatcctcatttactgtactggaagcagcaaccacctctgcaattttgcacaagaacctgagagtctgaaagacttttgttggcactggcctgtgttccactggggtggacacattaatctaaaagctaagaagctcacttcagctttgccaggcaaccagcacgtttgagcacaacggtgtagccaggaagggtgtgtgggaattttgatgctagtttttctttaattaggttgagagtgaaatcatggtctgtatgcatccaacaacttaatccaaaaagttggttgcacggctcctggaaggtttaccggcaaggaacctgtcccattttgtaagtaggtgggactggagagttgccaagaagcccccctccctgcccccttcctcctggcagattctgcttaaaatgagtgtggttcactgcagaatagtgtcacctgtcttcagatgtctaaagacataagaaaggcctagccagctaagtcagaaaaggggactttccggtgggaatgcattttccagtctctaggagaagtctttatccaaataaaattagcatagtggtggagtcactgccagggcgctggtgcggaagcacagcccagagccgaggacgtggccatcctccttcctctgatgcaaagtgttctctatccccagttaccatgagcaatatcagtagcatcgtgaaccgggcccgtgatgcctttaactcaggcaagactcgaccgctgcagttccgggttgagcagctggaggcgttgcagcgcatgatcaacgagaacttgaaaggcatctccaaggcgctggcctctaacctgcgcaaggtgcgttgggtgtggggagagagtggggctgagtggtgagtgggaggctacaagaagggactagcagggatacaacaggtacggggacagtcgcaccctgcgcgtagggcatcagttgccggccacccaattcaccgaggactactctgctcctcaaacactacatgtgtagtgcagtgttatagaagggagagtgaggctaggaaaggattagcaacgctttcagggtcaccttctccttccaccaccgttgaacagtctccttccttccgcctgtcgctttttctctatggccttggcttcttcactgaccccatttcaacagacccagccagccccactcccgccctcctgcctccgtcttccctggtctacctgacttgcataccaggaccaccaagatcgtgctcctgaacgaggcagctccatgtggcttcctctttcctcctgttccctgaggatgctctttccgtggctctgagaaaacccagcatcctccagatccacaagcctttcactttgctgtggggactcacctgagactgcttccaccctccttgtggcgccgcccaccctctgccctgtcaagactcactcaccaaactactgtctgtgcttcccccactttgggcaagaaagagaaaaattcactctttagacttcccgaagtccagcaaagagatgcataccccattggcgctccctctttcttgtccccttcccatcccaggaggatctgggttccccagcagccccagttcctgcctccctctgccatagttctggatttttgcctgccacgcttggtcacatcttcccgggtgggggttacacctgtgtctactcagcacggtggtctcccaagctttcttggattcgggtctcagtgtggtctgtaagcttccaacaatgcaatttcttccttaccgttctgggtcctgcctgtgccttctctatatcccacctccctgctatagttatgttaaacagccagctcaccaaggcaagcagagctttgattctttggtcctcaagttcacatggggcttctctctagaatgaatggacctcctactacgaggaggtggctcacgtgctggacgaaatcgatttcacgattaaggggctctccgattgggctgaggacgagcctgtggcaaagactcgtcagacccaggaagatgacctctacatccactcggagcccctgggcgtggtccttgtcataggtgcttggaactacccattcaacctcaccattcagcccatggtgggtgccatcgctgcaggtacgggggctccattctggagggactgttacaggggttgtgatggccaaaccgtctagccgggtctccaggctggcttctccatacttatggggagaggtgaagggtgaggactctccactatgcgtcaggagaggccagagccactctctcatccagcctgggcatctctgtgtgtgttctttcttctctcccagcctcccagccttgctgaggggcaggcggtgccgctctgacagcacatgccagtgcatgtacctgcagcagctgttcatgtgtgtacctgtgtgtgcttcagccttgtgttcctatatgtgtacccgtgcttctcagcgtgtgcctgcatgtactcacagcatgcacccatgtctagcctcccacccaaccttccactgccttccaaaggtacagatgataccgtggaatttctgggcctggtcactgggtgggaagagtctttgaggaggtctcctgaggagtcttctgactttgtattctgcaggaaatgctgtggtcctcaagccctcggaagtgagtgatcacatggcagacctgctttcaacacttatccctcagtatatggacaaggtaagcccccccccccgcccccagctcagggatgactgtgtgtgtgtgtggggggggtcaggtctttaaatccagggagacagtggattattattgcaaatgggaggagtccagcttctaacaacaggttagatggaacctttgagctaggagagtgacttacctgctagggtgcccatgtaagagctgaggggcagccagtctcccattggccattctatgtgtggagttgggagaggcagggtctttgctgagcctcctcggtctctttgctggaaggatctgtacccagtgatcaaagggggtgtccctgaaaccacggagctgctcaaagagaagtttgaccacatcatgtacacagggagcacggctgtaggaaagatcgtcatggcggctgccgccaagcatctgacccctgtcaccttggagcttggaggaaagagcccttgttacgtagacaaggactgtgatctagatgtggcttgccggtaaggagacttgggcttggccggctgcagaggcttggggcctgtcgacctttctaccttgctactaccatgatggtatcaagttttatggctgtaccgagtactaaagatggtccctgcccttagggagcctatagcgttgggatggtagggttggggggggcaccattcatagacgtcttcctagagggggcctccagagagagcaatgactctctggcattcctaggggtagtgatctaagtagaagctacatgaaaggctaatgtgggacactcagggcttagagccctttaccaagcccattgcccctctgcaggagatctcaggaaagccacttcgctcagagtctaggatgggactcacatctggactagcgaggcctctggtcatctctgtactacaaatatatggaggggcacaagtagaggtgtcacctggctgtgcaagactctgagaatgtaaccagagtcatagagtatttgccctagatgtccttagaatttcctcatgtcattttccacgacacactggttgcagaaagccctgtggtgccaggtggggtcgaccagctcagtgtgtctgtcagtgagttgttgcttaggaagatttgctgtggcagcccccagaccatgggccgtcctctagcactcagccttcagccgccttgcccccgaacatgtggatgttctcaaggggactcccatgatttacagactgtattttcacaggcgtattgcttggggaaagtttatgaacagtggccagacctgtgtggccccagactacatcctctgtgacccctccattcagaatgaaattgtggagaagctcaaaaagtcactgaaagtgagtatggcccgagggtgggggtaggaagctgtaaggcatgtgggctgcatttagcttcagccaacctccaggactctgaaaaatagtagctgtttatgaaatacaatgtggtgacagggtacctggtgaacctgtgccaaggtgtccccctgagaaattactccatgcaacggatctgatataaagagggtttattggggggcaagggagggcccagagaagaggtagaggcagacagatggcagcagagccatgagggcagaggacagaaagggggagagactgactaggaacacccaggaacagagagagagagagagagagagagagagacaaagatggaggagagctagggtgtcaagcagtccacgcctccagtacctggcagcagctgggtagtagcagtagatgatgactaaagctgttgctaggtacctgggaggagcccagcagaattgcctgtaagctaacagtgtttttcttctgctcacaaaagaaatatgcgcacacactgcagacaattgggaagggtacagagcataatgggtgtagggagtgagggaagctcttctcagattccctccacccagagggtgtcattgcctagggaataggaagtcaaggccatgatggggccaggccttccttgagtcctcatctcttctcaggatttctatggggaagatgctaagcagtcccatgactatgggaggatcatcaacgaccggcacttccagcgggtcataaatctgattgacagcaagaaagtagcccatggaggcacctgggatcagccttcacgatacataggtgtgtgttctgccattaagagtgctgcctgcttgcctggactgccagggtctacagcaagtttacaggaagtgggcacaggcactgagcaagtaggctttaccaagtaggcctgcagaaagctctggggccctcagaaggcaaacactgagcacggctcttaaggaatgcctctcgacagacctagaaacctccctgtcttccacaagtcagagacaagggcttcatctccacctccaatctgtgaaagggaacaggaaatggagccagcagaggccacaagagttcaaggcagaccaggccgggaggaactcccaggaccctggctgggggcaacatcagttggagttcttctggtccccagttaacaaggagacaggagaattatgggatgaatacatggggctggtagatataataactaccccttcagtgtttagagcctgttcccccatgcaggttaatctgtagatatccccatgatctgccaggatgtagtctaagctgcgggagttggggtaaagtgggtatgagaaagctttgtttgcaaaaggatagaggcaggagctagatgttggcactggctgtgtgtatatgtatgtgtgttggggatctggggtgggtcagcacaaacagactctggctcacttcctgcctcagctcccaccatcctagtggatgtggacccccagtccccggtgatgcaggaggagatctttgggccggtgatgcccattgtgtgtgttcgaagcttggatgaggccattaaatttatcaaccagcgcgagaagcccctggcactctatgtgttctccaacaatgacaaggtaggcctaggagtccccttcccagattcctggctcatcaggcacagcacttacatgcaagggggccccaggatccacaggcctgggttcagatttttaacttcctcattcattaggtgtgtgaccttgcaccaatcacttaacctgtctgcacttctttggccttatctggaagagtagaacagtcccatctttttacacctgtggatgaaatggccattgctatttccccatacagtgccaagggtcatgagacatttgcagagggctggagacatggcaggggctaaggatggctgagttggtggttggggctcagtctccactggtcccagttcttgagactccaagacatggtcagaatgggtgggaagaatgccagaatggcaacatggctgcctgggtctttacacaggtgatcaagaaaatgatcgcagagacatcaagcggtggagtgacagccaatgacgtcattgttcacatcactgtgcccactttgccctttgggggtgtgggtaagtctggtgctagtcacctggtactctggcctttcacaggggtcccaagcttggggtcacacaaccatttaatagcatgactggagtgagactctctctggacactcttgtgcctgacagagctgccctaggggacactctaagctttagctaaccccagaaggctcagaaaaagggattcatgaggactgagagtacacgattggctgggatcctttcatttagggaacagcggcatgggggcctaccatggcaagaaaagcttcgagaccttttcccaccgccgctcttgcctggtgaggtctctgcggaatgaagaagctaacaaggccaggtatcccccaagcccagccaaggtgagagacaaggccctggggattgggaaatgtggactgtgcaggactaggggtgaggccttgggtctcatgttctctccctgtcctagcagatgccccggcattgagagggctacttcacctgacacttgttttgctggctgtcctgtccttgaagaattgctcacggagcctcatcctggcttctctcccacctgctgccccatgcatgctcaactacccgaaggctctcacctcccccctcttgttctgctgtttgctgagcacagagaccaataaaaaagcctctaaag

SEQ ID NO: 7 - protein, 285 aa, Rattus norvegicus
MFLLTPVLVAVVCILVIWVFKNADRSLEEKKEEARAQPWVDEDLKDNTEHLQVEEDTEEWQESEESVEHILFSHTRYPEQEMRMRSQEFYELLSKRRSIRFISSEPVPMEVIDNVIKAAGTAPSGAHTEPWTFVVVKDPDMKHKIREIIEEEEEINYMKRMGKRWVTDLKKLRTNWIKEYLDTAPVLILIFKQVHGFAVNGKKKVHYYNEISVSIACGILLAALQNAGLVTVTTTPLNCGPRLRVLLGRPSHEKLLVLLPVGYPSRGATVPDLKRKTLDQIMVTV

SEQ ID NO: 8 - protein, 289 aa, Homo sapiens
MYFLTPILVAILCILVVWIFKNADRSMEKKKGEPRTRAEARPWVDEDLKDSSDLHQAEEDADEWQESEENVEHIPFSHNHYPEKEMVKRSQEFYELLNKRRSVRFISNEQVPMEVIDNVIRTAGTAPSGAHTEPWTFVVVKDPDVKHKIRKIIEEEEEINYMKRMGHRWVTDLKKLRTNWIKEYLDTAPILILIFKQVHGFAANGKKKVHYYNEISVSIACGILLAALQNAGLVTVTTTPLNCGPRLRVLLGRPAHEKLLMLLPVGYPSKEATVPDLKRKPLDQIMVTV

SEQ ID NO: 9 - protein, 277 aa, Homo sapiens
MYFLTPILVAILCILVVWIFKNADRSMEKKKGEPRTRAEARPWVDEDLKDSSDLHQAEEEHIPFSHNHYPEKEMVKRSQEFYELLNKRRSVRFISNEQVPMEVIDNVIRTAGTAPSGAHTEPWTFVVVKDPDVKHKIRKIIEEEEEINYMKRMGHRWVTDLKKLRTNWIKEYLDTAPILILIFKQVHGFAANGKKKVHYYNEISVSIACGILLAALQNAGLVTVTTTPLNCGPRLRVLLGRPAHEKLLMLLPVGYPSKEATVPDLKRKPLDQIMVTV

SEQ ID NO: 10 - protein, 289 aa, Homo sapiens
MYFLTPILVAILCILVVWIFKNADRSMEKKKGEPRTRAEARPWVDEDLKDSSDLHQAEEDADEWQESEENVEHIPFSHNHYPEKEMVKRSQEFYELLNKRRSVRFISNEQVPMEVIDNVIRTAGTAPSGAHTEPWTFVVVKDPDVKHKIRKIIEEEEEINYMKRMGHRWVTDLKKLRTNWIKEYLDTAPILILIFKQVHGFAANGKKKVHYYNEISVSIACGILLAALQNAGLVTVTTTPLNCGPRLRVLLGRPAHEKLPMLLPVGYPSKEATVPDLKRKPLDQIMVTV

SEQ ID NO: 11 - protein, 289 aa, Pan troglodytes
MYFLTPILVAILCILVVWIFKNADRSMEKKKGEPRTRAEARPWVDEDLKDSSDLHQAEEDADEWQESEEKVEHIPFSHTHYPEKEMVKRSQEFYELLNKRRSVRFIRNEQVPMEVIDNVIRTAGTAPSGAHTEPWTFVVVKDPDVKHKIRKIIEEEEEINYMKRMGHRWVTDLKKLRTNWIKEYLDTAPILILIFKQVHGFATNGKKKVHYYNEISVSIACGILLAALQNAGLVTVTTTPLNCGPRLRVLLGRPAHEKLLMLLPVGYPSKEATVPDLRRKPLDQIMVTV

SEQ ID NO: 12 - protein, 289 aa, Pongo pygmaeus
MYFLTPILVAILCILVVWIFKNADRSMEKKKGEARTRAEARPWVDEDLKDSSDLHQAEEDADEWQESEENVEHIPFSHTHYPEKEMVKRSREFYELLNKRRSVRFISNEQVPMEVIDNVIRTAGTAPSGAHTEPWTFVVVKDPDVKHKIRKIIEGEEEINYMKRMGHRWVTDLKKLRTNWIKEYLDTAPILILIFKQVHGFVANGKKKVHYYNEISVSIACGILLAALQNAGLVTVTTTPLNCGPRLRVLLGRPAHEKLLMLLPVGYPSKEAMVPDLKRKPLDQIMVMV

SEQ ID NO: 13 - protein, 289 aa, Sus scrofa
MFLLTPVLVAVVCILMVWIFKNADGATKRREEEPRARAEARPWVDEDLKDSTDVLQVEEDADEWQESEEEVEHVPFSHTRYPEKEMVRRSQEFYELLNKRRSVRFISNERVPMEVIDNVIKAAGTAPSGAHTEPWTFVVVKDPDVKHRIREIMEEEEKINYLKRMGPRWVTDLKKLRTNWIKEYWDTAPVLILIFKQVHGFAANGKKKIHYYNEISVSIACGIFLAALQNAGLVTVTTTPFNCGPRLRVFLNRPANEKLLMFLPVGYPSKEATVPDLPRKPLDQIMVTV

SEQ ID NO: 14 - protein, 453 aa, Mus musculus
MSNISSIVNRARDAFNSGKTRPLQFRVEQLEALQRMINENLKGISKALASNLRKNEWTSYYEEVAHVLDEIDFTIKGLSDWAEDEPVGKTRQTQEDDLYIHSEPLGVVLVIGAWNYPFNLTIQPMVGAIAAGNAVVLKPSEVSDHMADLLSTLIPQYMDKDLYPVIKGGVPETTELLKEKFDHIMYTGSTAVGKIVMAAAAKHLTPVTLELGGKSPCYVDKDCDLDVACRRIAWGKFMNSGQTCVAPDYILCDPSIQNEIVEKLKKSLKDFYGEDAKQSHDYGRIINDRHFQRVINLIDSKKVAHGGTWDQPSRYIAPTILVDVDPQSPVMQEEIFGPVMPIVCVRSLDEAIKFINQREKPLALYVFSNNDKVIKKMIAETSSGGVTANDVIVHITVPTLPFGGVGNSGMGAYHGKKSFETFSHRRSCLVRSLRNEEANKARYPPSPAKMPRH

SEQ ID NO: 15 - protein, 453 aa, Mus musculus
MSNISSIVNRARDAFNSGKTRPLQFRVEQLEALQRMINENLKGISKALASNLRKNEWTSYYEEVAHVLDEIDFTIKGLSDWAEDEPVAKTRQTQEDDLYIHSEPLGVVLVIGAWNYPFNLTIQPMVGAIAAGNAVVLKPSEVSDHMADLLSTLNPQYMDKDLYPVIKGGVPETTELLKEKFDHIMYTGSTAVGKIVMAAAAKHLTPVTLELGGKSPCYVDKDCDLDVACRRIAWGKFMNSGQTCVAPDYILCDPSIQNEIVEKLKKSLKDFYGEDAKQSHDYGRIINDRHFQRVINLIDSKKVARGGTWDQPSRYIAPTILVDVDPQSPVMQEEIFGPVMPIVCVRSLDEAIKFINQREKPLALYVFSNNDKVIKKMIAETSSGGVTANDVIVHITVPTLPFGGVGNSGMGAYHGKKSFETFSHRRSCLVRSLRNEEANKARYPPSPAKMPRH

SEQ ID NO: 16 - protein, 453 aa, Mus musculus
MSNISSIVNRARDAFNSGKTRPLQFRVEQLEALQRMINENLKGISKALASNLRKNEWTSYYEEVAHVLDEIDFTIKGLSDWAEDEPVRKTRQTQEDDLYIHSEPLGVVLVIGAWNYPFNLTIQPMVGAIAAGNAVVLKPSEVSDHMADLLSTLNPQYMDKDLYPVIKGGVPETTELLKEKFDHIMYTGSTAVGKIVMAAAAKHLTPVTLELGGKSPCYVDKDCDLDVACRRIAWGKFMNSGQTCVAPDYILCDPSIQNEIVEKLKKSLKDFYGEDAKQSHDYGRIINDRHFQRVINLIDSKKVARGGTWDQPSRYIAPTILVDVDPQSPVMQEEIFGPVMPIVCVRSLDEAVKFINQREKPLALYVFSNNDKVIKKMIAETSSGGVTANDVIVHITVPTLPFGGVGNSGMGAYHGKKSFETFSHRRSCLVRSLRNEEANKARYPPSPAKMPRH

SEQ ID NO: 17 - protein, 453 aa, Rattus norvegicus
MSSISDTVKRAREAFNSGKTRSLQFRIQQLEALQRMINENLKSISGALASDLGKNEWTSYYEEVAHVLEELDTTIKELPDWAEDEPVAKTRQTQQDDLYIHSEPLGVVLVIGAWNYPFNLTIQPMVGAVAAGNAVILKPSEVSGHMADLLATLIPQYMDQNLYLVVKGGVPETTELLKERFDHIMYTGSTAVGKIVMAAAAKHLTPVTLELGGKSPCYVDKDCDLDVACRRIAWGKFMNSGQTCVAPDYILCDPSIQNQIVEKLKKSLKDFYGEDAKQSRDYGRIINDRHFQRVKGLIDNQKVAHGGTWDQSSRYIAPTILVDVDPQSPVMQEEIFGPVMPIVCVRSLEEAIQFINQREKPLALYVFSNNEKVIKKMIAETSSGGVTANDVIVHITVPTLPFGGVGNSGMGAYHGKKSFETFSHRRSCLVKSLLNEEAHKARYPPSPAKMPRH

SEQ ID NO: 18 - protein, 452 aa, Rattus norvegicus
MSISDTVKRAREAFNSGKTRSLQFRIQQLEALQRMINENLKSISGALASDLGKNEWTSYYEEVAHVLEELDTTIKELPDWAEDEPVAKTRQTQQDDLYIHSEPLGVVLVIGAWNYPFNLTIQPMVGAVAAGNAVILKPSEVSGHMADLLATLIPQYMDQNLYLVVKGGVPETTELLKERFDHIMYTGSTAVGKIVMAAAAKHLTPVTLELGGKSPCYVDKDCDLDVACRRIAWGKFMNSGQTCVAPDYILCDPSIQNQIVEKLKKSLKDFYGEDAKQSRDYGRIINDRHFQRVKGLIDNQKVAHGGTWDQSSRYIAPTILVDVDPQSPVMQEEIFGPVMPIVCVRSLEEAIQFINQREKPLALYVFSNNEKVIKKMIAETSSGGVTANDVIVHITVPTLPFGGVGNSGMGAYHGKKSFETFSHRRSCLVKSLLNEEAHKARYPPSPAKMPRH

SEQ ID NO: 19 - protein, 453 aa, Homo sapiens [sequence omitted]

SEQ ID NO: 20 - protein, 453 aa, Homo sapiens
MSKISEAVKRARAAFSSGRTRPLQFRIQQLEALQRLIQEQEQELVGALAADLHKNEWNAYYEEVVYVLEEIEYMIQKLPEWAADEPVEKTPQTQQDELYIHSEPLGVVLVIGTWNYPFNLTIQPMVGAIAAGNSVVLKPSELSENMASLLATIIPQYLDKDLYPVINGGVPETTELLKERFDHILYTGSTGVGKIIMTAAAKHLTPVTLELGGKSPCYVDKNCDLDVACRRIAWGKFMNSGQTCVAPDYILCDPSIQNQIVEKLKKSLKEFYGEDAKKSRDYGRIISARHFQRVMGLIEGQKVAYGGTGDAATRYIAPTILTDVDPQSPVMQEEIFGPVLPIVCVRSLEEAIQFINQREKPLALYMFSSNDKVIKKMIAETSSGGVAANDVIVHITLHSLPFGGVGNSGMGSYHGKKSFETFSHRRSCLVRPLMNDEGLKVRYPPSPAKMTQH

SEQ ID NO: 21 - protein, 453 aa, Homo sapiens
MSKISEAVKRARAAFSSGRTRPLQFRFQQLEALQRLIQEQEQELVGALAADLHKNEWNAYYEEVVYVLEEIEYMIQKLPEWAADEPVEKTPQTQQDELYIHSEPLGVVLVIGTWNYPFNLTIQPMVGAIAAGNAVVLKPSELSENMASLLATIIPQYLDKDLYPVINGGVPETTELLKERFDHILYTGSTGVGKIIMTAAAKHLTPVTLELGGKSPCYVDKNCDLDVACRRIAWGKFMNSGQTCVAPDYILCDPSIQNQIVEKLKKSLKEFYGEDAKKSRDYGRIISARHFQRVMGLIEGQKVAYGGTGDAATRYIAPTILTDVDPQSPVMQEEIFGPVLPIVCVRSLEEAIQFINQREKPLALYMFSSNDKVIKKMIAETSSGGVAANDVIVHITLHSLPFGGVGNSGMGSYHGKKSFETFSHRRSCLVRPLMNDEGLKVRYPPSPAKMTQH

SEQ ID NO: 22 - protein, 453 aa, Homo sapiens [sequence omitted]

SEQ ID NO: 23 - protein, 471 aa, Canis familiaris
MEGESSTGTPGGKVGTGTMSKISEVVQRARAAFNSGKTRPLQFRIQQLEALRRMIKEHEKDLAGALTADLHKNEWNAYYEEVVYVLEEIEYMIKKLPEWAADEPVEKTPQTQQDECYIHSEPLGVVLIIGTWNYPFTVTIQPMVGAIAAGNAVVIKPSELSENMANLLATIIPQYLDRDLYPVISGGIPETTELLKERFDHILYTGNTAVGKVIMMAAAKHLTPVTLELGGKNPCYVDKDCDLDIACRRIAWGKFMNSGQTCVAPDYILCDPSIQNQIVEKLKKALKEFYGEDAKKSRDYGRIINSRHFQRVMGLMEGQKVAYGGTGDAATRYIAPTILIDVDTQSQVMQEEIFGPVMPIVCVRSLEEAIQFINQREKPLALYVFSLNDKMIKKMIAETSSGGVTANDVIVHVSVHSLPYGGVGNSGMGSYHGKKSFETFSHCRSCLVRPLLNDESLKTRYPPSLAKMTRH

SEQ ID NO: 24 - protein, 453 aa, Bos taurus
MSAISEVVQRARAAFNSGRTRPLQFRVQQLEGLRRLIREREKDLVGALAADLHKNEWTAYYEEIVYVLEEIDYMIRKLPEWAADEPVEKTPHTQQDEAYIHSEPLGVVLIIGSWNYPFNLTIQPMVGAIAAGNAVVLKPSELSENTASLLATILPQYLDQDLYPVINGGVAETTEVLKERFDHILFTGSTGVGRVVMMAAAKHLTPVTLELGGKNPCYVDKDCDLDIACRRIAWGKFMNSGQTCVAPDYILCDPSIQSQVVEKLKKSLKEFYGEDAKKSRDYGRIINSRHFQRVMGLLEGQKVTYGGTGDATTRYIAPTILTDVDPESPVMQEEVFGPVLPIMCVRSLEEAIQFITQREKPLALYVFSPNDKVIKKMIAETSSGGVTANDVVVHISVHSLPYGGVGDSGMGSYHGRKSFETFSHRRSCLVRPLLNEETLKARYPPSPAKMPRH

(19) United States
(12) Patent Application Publication
(10) Pub. No.: US 2006/0263880 A1
(43) Pub. Date: Nov. 23, 2006

(54) Protein factors supporting undifferentiated growth of human embryonic stem cells

(75) Inventor: G. Rasul Chaudhry, Rochester Hills, MI (US)

Correspondence Address: Ian C. McLeod;McLeod & Moyne, P.C., 2190 Commons Parkway, Okemos, MI, 48864, US

(73) Assignee: Oakland University, Rochester, MI (US)

(21) Appl. No.: 11/435,204

(22) Filed: May 16, 2006

Related U.S. Application Data
(60) Provisional application No. 60/682,559, filed on May 19, 2005.

(51) Int. Cl. C12N 5/08; C12N 5/02

(52) U.S. Cl. 435/366; 435/404

(57) ABSTRACT

Factors which are secreted by mouse embryonic fibroblasts which promote maintenance and support the growth of human embryonic stem (hES) cells are provided. The factors can be isolated by chromatographic techniques. The genes, cDNA and amino acid sequences for the factors are disclosed herein and identified. The cDNA of one factor encodes a deduced nitroreductase family containing protein with 285 amino acids and a mass of approximately 33 kD. This factor is identified as iodotyrosine dehalogenase. A second factor supporting undifferentiated hES cell growth is a 453 amino acid encoded by an aldehyde dehydrogenase gene. The use of recombinant factors can eliminate the need for an MEF feeder layer for growing hES cells without differentiation.

### CROSS-REFERENCE TO RELATED APPLICATIONS

This application claims benefit of Provisional Application No. 60/682,559, filed May 19, 2005.

### STATEMENT REGARDING FEDERALLY SPONSORED RESEARCH OR DEVELOPMENT

Not Applicable

### BACKGROUND OF THE INVENTION

(1) Field of the Invention

The present invention relates generally to supporting the undifferentiated growth of human embryonic stem cells, and more particularly to factors which support undifferentiated growth of stem cells. Specifically, the present invention relates to protein factors which support undifferentiated growth.

(2) Description of the Related Art

Embryonic stem (ES) cells are derived from the inner cell mass of the blastocyst. They can be grown as undifferentiated pluripotent cells in vitro in specialized medium. While leukemia inhibitory factor (LIF) supports undifferentiated growth of mouse ES (mES) cells in the culture medium, human ES (hES) cells require a feeder layer of mouse embryonic fibroblasts (MEFs) for their undifferentiated growth. The cultivation of hES cells on a MEF feeder layer is time-consuming and requires maintenance of two cell cultures simultaneously. Therefore, much interest has been shown in devising an efficient protocol for growing hES cells without a feeder layer.

U.S. Pat. No. 6,642,048 to Xu et al. teaches a method of proliferating human stem cells in a substantially undifferentiated form. Xu et al. discloses culture media essentially free of feeder cells which is conditioned by certain cell lines such as immortalized mouse cell lines, human cell lines, mesenchymal cells and fibroblasts.

U.S. Patent Application Publication No. 2003/0143736 A1 and 2004/0253721 A1 to Bongso et al. discloses methods of propagating undifferentiated hES cells on human feeder layers and/or in the absence of a feeder layer. The feeder layers can be human fetal muscle, human fetal skin, human adult fallopian tube fibroblasts and human adult skin cells.

U.S. Patent Application Publication No. 2004/0235159 A1 to Mandalam et al. discloses a fresh medium for culturing hES cells having components which support proliferation without differentiation. The medium can be added fresh without preconditioning by another cell type.

U.S. Patent Application Publication No. 2005/0037488 A1 to Mitalipova et al. discloses methods for the culture of hES cells with human granulosa feeder cells, muscle cells, Fallopian ductal epithelial cells, bone marrow stromal cells, and skin fibroblasts.

While the related art teach support of human embryonic stem cells using a feeder layer of mouse embryonic fibroblasts, there still exists a need for a factor(s) which supports undifferentiated hES growth.

### OBJECTS

Therefore, it is an object of the present invention to support undifferentiated growth of human embryonic stem cells without MEFs.

It is further an object of the present invention to provide factors which support the undifferentiated growth of human embryonic stem cells.

These and other objects will become increasingly apparent by reference to the following description.

### SUMMARY OF THE INVENTION

The present invention provides a medium for culturing stem cells while maintaining the stem cells in an undifferentiated state comprising one or more polypeptides homologous to iodotyrosine dehalogenase or aldehyde dehydrogenase. In further embodiments, the one or more polypeptides are recombinant.

The present invention provides a method of culturing stem cells while maintaining the stem cells in an undifferentiated state comprising culturing the stem cells in a medium comprising one or more polypeptides homologous to iodotyrosine dehalogenase or aldehyde dehydrogenase.

In further embodiments, the stem cells are human stem cells. In still further embodiments, the human stem cells are human embryonic stem cells. In still further embodiments, the one or more polypeptides are recombinant.

The present invention provides a method of culturing stem cells while maintaining the cells in an undifferentiated state comprising culturing the stem cells in a medium comprising one or more polypeptides having an amino acid sequence essentially identical to the amino acid sequences set forth in SEQ ID NO: 1 or SEQ ID NO: 4.

In further embodiments the stem cells are human stem cells. In still further embodiments the human stem cells are human embryonic stem cells. In still further embodiments the one or more polypeptides are recombinant.

The present invention provides a method of preparing a partially purified conditioned medium for culturing stem cells while maintaining the cells in an undifferentiated state comprising: providing a medium; conditioning the medium by culturing fibroblasts in the medium; precipitating the conditioned medium with a first salt solution to provide a first set of fractions; selecting a fraction of the first set of fractions of the precipitated conditioned medium; applying the selected fraction to a cation-exchange resin; eluting a second set of fractions with a second salt solution; and selecting a fraction of the second set of fractions as the partially purified conditioned medium for culturing stem cells.

In further embodiments the fibroblast are mouse embryonic fibroblasts. In still further embodiments the second salt solution is a 500 mM potassium chloride solution.

The present invention provides a partially purified conditioned medium for culturing stem cells while maintaining the cells in an undifferentiated state prepared by the method comprising: providing a medium; conditioning the medium by culturing fibroblasts in the medium; precipitating the conditioned medium with a first salt solution to provide a first set of fractions; selecting a fraction of the first set of fractions of the precipitated conditioned medium; applying the selected fraction to a cation-exchange resin; eluting a second set of fractions with a second salt solution; and selecting a fraction of the second set of fractions as the partially purified conditioned medium for culturing stem cells.

In further embodiments the fibroblast are mouse embryonic fibroblasts. In still further embodiments the second salt solution is a 500 mM potassium chloride solution.

The present invention provides a method of culturing stem cells while maintaining the cells in an undifferentiated state comprising: providing stem cells for culturing in an undifferentiated state; providing partially purified conditioned medium for culturing stem cells while maintaining the cells in an undifferentiated state prepared by the method comprising: providing a medium; conditioning the medium by culturing fibroblasts in the medium; precipitating the conditioned medium with a salt to provide a first set of fractions; selecting one fraction of the first set of fractions of the precipitated conditioned medium; applying the selected fraction to a cation-exchange resin; eluting a second set of fractions; and selecting one of the second set of fractions as the partially purified conditioned medium to a culture of stem cells; and culturing the stem cells in a medium comprising a fresh culture medium and the partially purified conditioned medium.

### BRIEF DESCRIPTION OF THE DRAWINGS

FIG. 1 is a light microscopy photograph of a differentiating four day old hES colony grown on MATRIGEL® substrate with faction I.

FIG. 2 is a light microscopy photograph of a differentiating two day old hES colony growing on a MATRIGEL® substrate-coated plate containing medium supplemented with faction III.

FIG. 3 is a light microscopy photograph of a differentiating two day old hES colony growing on the feeder layer.

FIG. 4 is an SDS-PAGE analysis of fractionation of conditioned medium. Shown are lane 1, fraction I; lane 2, faction II; lane 3, fraction III; and lane 4 conditioned medium.

FIG. 5 is an SDS-PAGE analysis of column elute. Shown are lane 1, fraction III; lane 2, fraction five (5).

FIG. 6 is the protein view of the Mascot® search engine results (Matrix Science Inc., Boston, Mass.) of a factor from Example 1 subjected to LC/MS/MS analysis using nano-spray LC/MS/MS mass spectrometer later identified as iodotyrosine dehalogenase.

FIG. 7 is the protein view of the Mascot® search engine results (Matrix Science Inc., Boston, Mass.) of another factor from Example 1 subjected to LC/MS/MS analysis using nano-spray LC/MS/MS mass spectrometer identified as aldehyde dehydrogenase.

### DETAILED DESCRIPTION OF THE INVENTION

All patents, patent applications, government publications, government regulations, and literature references cited in this specification are hereby incorporated herein by reference in their entirety. In case of conflict, the present description, including definitions, will control.

The term “conditioned medium” as used herein refers to hES medium which has been incubated with an MEF feeder layer.

The term “ES cells” as used herein refers to embryonic stem cells. The term “hES cells” as used herein refers to human embryonic stem cells.

The term “essentially identical” as used herein refers to any homologous sequence that is at least 98% identical to that of a given sequence.

The term “homologous” as used herein refers to a protein having a polypeptide sequence that is at least 80% identical to that of a given protein. Some homologous proteins to the mouse iodotyrosine dehalogenase include rat (SEQ ID NO: 7), human (SEQ ID NOs: 8, 9, 10), chimpanzee (SEQ ID NO: 11), orangutan (SEQ ID NO: 12) and pig (SEQ ID NO: 13) proteins. Some homologous proteins to the mouse aldehyde dehydrogenase include other mouse (SEQ ID NOs: 14, 15, 16), rat (SEQ ID NOs: 17, 18), human (SEQ ID NOs: 19-22), dog (SEQ ID NO: 23) and cattle (SEQ ID NO: 24) proteins.

The term “recombinant” as used herein refers to polypeptides that are obtained from genetically modified host organisms having one or more recombinant DNAs encoding the amino acid sequence of the polypeptide.

In 1998 human embryonic stem (hES) cells capable of growing in vitro were isolated from early human embryos (Thomson J A et al., Science November 6; 282(5391): 1145-7, 1998). These cells have shown remarkable ability to self renew and differentiate into various cell types under selective growth conditions. However, maintenance and culturing of hES cells are technically challenging. While leukemia inhibitory (LIF) (Brook and Gardner, Proc Natl Acad Sci USA. May 27; 94(11):5709-12, 1997) supports undifferentiated growth of mouse ES (mES) cells in the culture medium, hES cells require a feeder layer of mouse embryonic fibroblasts (MEFs) for their undifferentiated growth (Passier and Mummery, Cardiovasc Res. May 1; 58(2): 324-35, 2003). LIF is a 37-kilodalton (kD) cytokine produced by the MEFs (Sato et al., Nat Med. 10(1): 55-63 2004; Amit et al., Biol Reprod. November 2003; Smith, Hussain and Chaudhry, Meeting of Minds Journal of Undergraduate Research 2003; and Chaudhry et al., Osteogenic Cells Derived From Embryonic Stem Cells Produced Bone Nodules in Three-Dimensional Scaffolds, J. Biomed Biotechnol. 2004; 2004(4):203-210.). However, neither mouse nor human LIF supports the undifferentiated growth of hES cells.

Studies have shown that hES cells can be cultured on MATRIGEL® substrate (BD Discovery Labware, Inc., Bedford, Mass.) coated plates in the presence of conditioned medium which is hES cell medium incubated with the MEF feeder layer (Rosler et al., Dev Dyn. February; 229(2):259-74, 2004). These observations lead us to hypothesize that the conditioned medium contains one or more factors secreted by the MEF feeder layer which promote maintenance and support the growth of hES cells which can be isolated and identified by chromatographic techniques. Subsequently, the genes for the one or more factors can be cloned to produce recombinant factors in large amounts. The use of recombinant factors should eliminate the need of the MEF feeder layer for growing hES cells.

These observations prompted us to analyze the conditioned medium for the possibility of separating the one or more components of the conditioned medium that support undifferentiated growth of hES cells in vitro. The conditioned medium was fractionated using salt precipitation. The fractions were then tested for supporting growth of hES cells on MATRIGEL®-coated plates in the absence of a feeder layer. The results from these experiments showed that one of the factions supported undifferentiated growth of hES cells. The fraction showing undifferentiated growth was further subjected to DEAE column chromatography, analyzed by SDS-PAGE and tested for maintenance and growth of hES cells. The hES cells grown in the presence of the one or more isolated factors were analyzed for ES cell markers. This approach can lead to the identification of a LIF-like components in the conditions medium that would accelerate progress in hES cell research.

### EXAMPLE 1

Stem cell culture: Human ES cells (WA01) were maintained and cultured as previously described (Sperger et al., PNAS November; 100(23):13350-13355, 2003). Briefly, the cells were grown in six-well culture plates coated with 0.1 percent (%) gelatin (Sigma, St. Louis, Mo.) for a minimum of twenty-four hours (24 h) prior to experimentation. The human ES cell growth medium was prepared with 80% Dulbecco's Modified Eagle's Medium: Nutrient Mix F-12 (DMEM/F-12), 20% Knockout SR, and 1% L-glutamine (Invitrogen, Carlsbad, Calif.) dissolved in Phosphate Buffered Saline (PBS) supplemented with 2-Mercaptoethanol (0.001% v/v) (Sigma), 1% Modified Eagle Medium (MEM) Non-Essential Amino Acids Solution (Invitrogen), and 4 ng/ml of basic fibroblast growth factor (Invitrogen). The cell cultures were incubated in a 37° C. incubator with an atmosphere of 5% CO₂. The hES cells growth was examined daily by light microscopy.

Culture of feeder layer: The feeder layer MEFs were isolated from 13-day-old mouse embryos of strain CF1 (Kaufman et al., J Embryol Exp Morphol. February; 73: 249-61, 1983) and cultured in 75 cm² flasks to 70-80% confluency using growth medium consisting of 90% DMEM, 10% heat-inactivated fetal bovine serum, and 1% MEM Non-Essential Amino Acids Solution. Cells were harvested by trypsin-EDTA (Invitrogen) treatment and counted by hemacytometer. Appropriate amount of cell suspension was irradiated to an appropriate dose. Irradiated cells were plated on the gelatin coated 6-well culture plates.

Conditioned medium: The irradiated MEF feeder layer was incubated with hES cell culture medium (2.5 ml per well) overnight at 37° C. with 5% CO₂.

Fractionation of the conditioned medium: The conditioned medium (500 ml) was fractionated by salt precipitation using ammonium sulfate into three separate fractions (I, II and III). Fraction I was obtained by precipitation with a fifty percent (50%) ammonium sulfate solution. Fraction II was obtained by precipitation with an eighty percent (80%) ammonium sulfate solution. Fraction III was obtained by precipitation with a one hundred percent (100%) ammonium sulfate solution. The fractions were dialyzed against 10 mM Tris-HCl, pH. 7.5 and stored at 4° C. and tested for supporting the hES cell growth in the absence of a feeder layer.

Testing of fractions: The fractions were spiked into the medium in the MATRIGEL®-coated plates. The medium was changed and supplemented with the respective fractions every other day and cell growth was monitored by light microscopy (LM). One of the fractions (fraction III) supporting the undifferentiated cell growth of hES cells was subjected to further chromatographic analysis.

Column chromatography: The fraction III was applied to a preformed DEAE (DE52 diethylaminoethyl-cellulose, Whatman, Brentford, UK) column and eluted with 50 mM Tris-HCl, pH 7.5 containing 10 mM, 100 mM or 500 mM KCl. The eluants (1-2 ml) were collected, stored at 4° C. and further analyzed.

Sodium dodecyl sulfate polyacrylamide gel electrophoresis (SDS-PAGE): The fractions were subjected to SDS-PAGE using 12% separating gel and 2.5% spacing gel following the Bio-Rad (Hercules, Calif.) protocol. The proteins were visualized by Coomassie blue (Fisher Scientific, Hampton, N.H.) staining of the gel followed by destaining with 50% methanol/40% water/10% acetic acid.

Conditions for MEF growth and production of conditioned medium: MEF cells were grown in 75 cm² flasks. A confluency of 70-80% was achieved upon two to three (2-3) days of incubation as previously described. MEF were routinely used as feeder layers up to five (5) passages. MEF beyond five (5) passages did not support undifferentiated hES cell growth. MEF were irradiated at various levels (10-65 Gy) of gamma irradiation. The irradiated MEF were grown in MEF medium and monitored by light microscopy (LM). This screening led us to select a radiation dose of 55 Gy for routine treatment of MEFs used as a feeder layer for maintenance and culturing of hES cells. In order to determine the appropriate amount of MEFs supporting the hES cell growth different concentrations (1×10⁴-1×10⁶ cells per milliliter) the MEFs were tested. A concentration of 0.75×10⁵ cells per milliliter (ml) was found to be optimally supporting the growth of hES cells. The radiation dose and the concentration of MEFs supporting the optimal growth of hES cells were similar to those previously reported. (Levenberg et al. PNAS April; 99 (7): 4391-4396, 2002).

In order to produce conditioned medium capable of supporting hES cells irradiated MEFs were plated as feeder layer in six-well cell culture plates and incubated with the hES cell medium as described. After overnight incubation the medium was collected and fresh hES cell medium was added to the wells daily for up to fourteen (14) days. The collected medium was termed the conditioned medium and frozen at −20° C. until used. The frozen conditioned medium was thawed and analyzed further for the factor(s) supporting the hES cell growth.

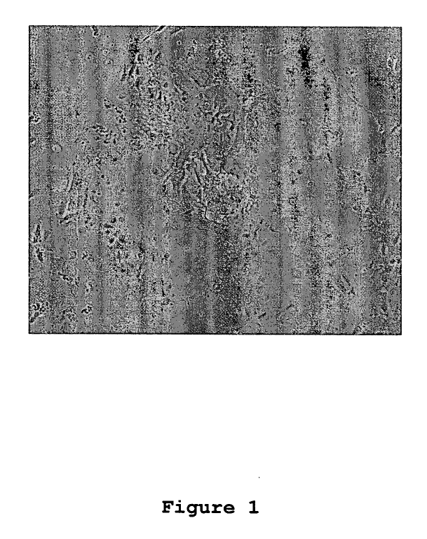

Analysis of conditioned medium: The conditioned medium was first fractionated by salt precipitation into three fractions (I, II, and III) with varying salt saturations. Human ES cells were grown in the presence of each fraction after dialysis and filtration by a 0.2 μm filter. Simultaneously, positive and negative controls contained MEF feeder layer and hES cell culture medium only. When fractions I or II were supplemented with the hES cell medium the hES cells were unable to maintain undifferentiated growth. FIG. 1 shows the growth of hES cells in the hES cell medium supplemented with fraction I.

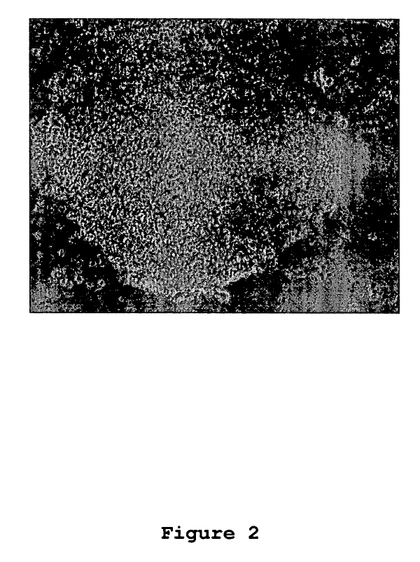

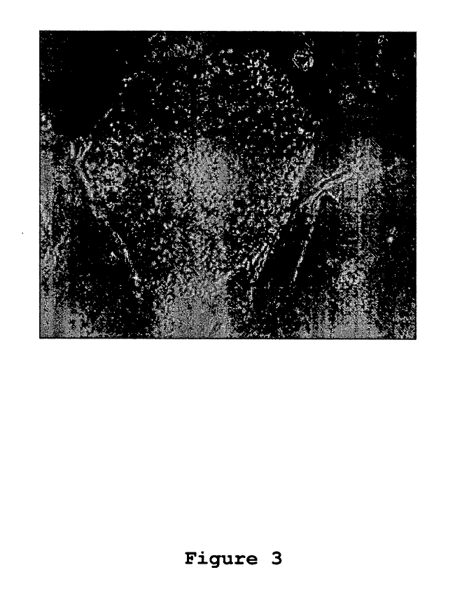

Analysis of conditioned medium: The conditioned medium was first fractionated by salt precipitation into three fractions (labeled with Roman numerals: I, II, and III) with varying salt saturations as shown in Table 1. Human ES cells were grown in the presence of each fraction after dialysis and filtration by a 0.2 μm filter. Simultaneously, positive and negative controls contained MEF feeder layer and hES cell culture medium only. The results of these experiments showed that fraction III supported the growth of hES cells as shown in FIG. 2, similar to the MEF feeder layer as shown in FIG. 3.

Based on these results, fraction III was selected for further studies. Fraction III was applied to a preformed DEAE column and eluted with different concentrations of potassium chloride (KCl) as seen in Table 2. The fractions eluted from the DEAE column (labeled with Arabic numerals: 1-8) were tested for supporting the hES cell growth and analyzed by SDS-PAGE. These results demonstrated that only fraction five (5) supported the growth of hES cells.

TABLE 1
Fractionation of the conditioned medium and
identification of the fraction supporting hES cell growth.

| Treatment | hES cell growth |
|---|---|
| MEFs feeder layer | Positive |
| Conditioned medium | Positive |
| Fraction I | Negative |
| Fraction II | Negative |
| Fraction III | Positive |

TABLE 2
DEAE column chromatography of the fraction III and
growth of hES cells in the medium containing the isolated
factors.

| Fraction | Description | hES cell growth |
|---|---|---|
| 1 | Eluted with 10 mM KCl | Negative |
| 2 | Eluted with 10 mM KCl | Negative |
| 3 | Eluted with 100 mM KCl | Negative |
| 4 | Eluted with 100 mM KCl | Negative |
| 5 | Eluted with 500 mM KCl | Positive |
| 6 | Eluted with 500 mM KCl | Negative |
| 7 | Eluted with 500 mM KCl | Negative |
| 8 | Eluted with 500 mM KCl | Negative |

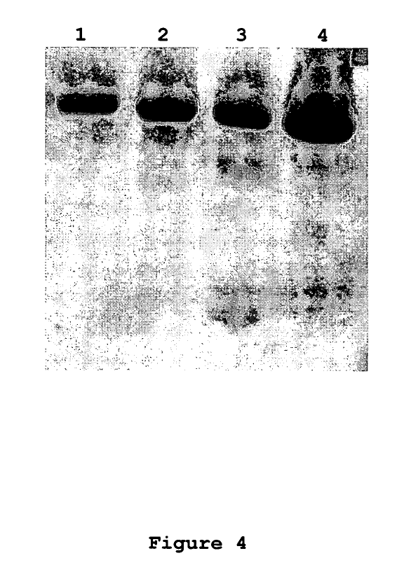

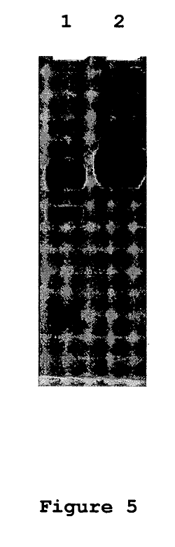

The results of the DEAE column fractions analyzed by SDS-PAGE are shown in FIG. 4 and FIG. 5. It is evident that a common band in the range of 66 kD (bovine serum albumin, BSA) was difficult to be separated because of its presence in higher concentrations in the medium. Several smaller bands appeared in fraction III, which showed support for undifferentiated hES cells. Fraction five (5) of the DEAE column had two bands that are evident in the fraction III as can be seen in FIG. 5. These bands suggest a factor ranging from 20,000-40,000 kD may be involved in supporting the hES cell growth. This Example demonstrates that the conditioned medium can be fractionated to isolate one or more factors that support undifferentiated growth of hES cells.

### EXAMPLE 2

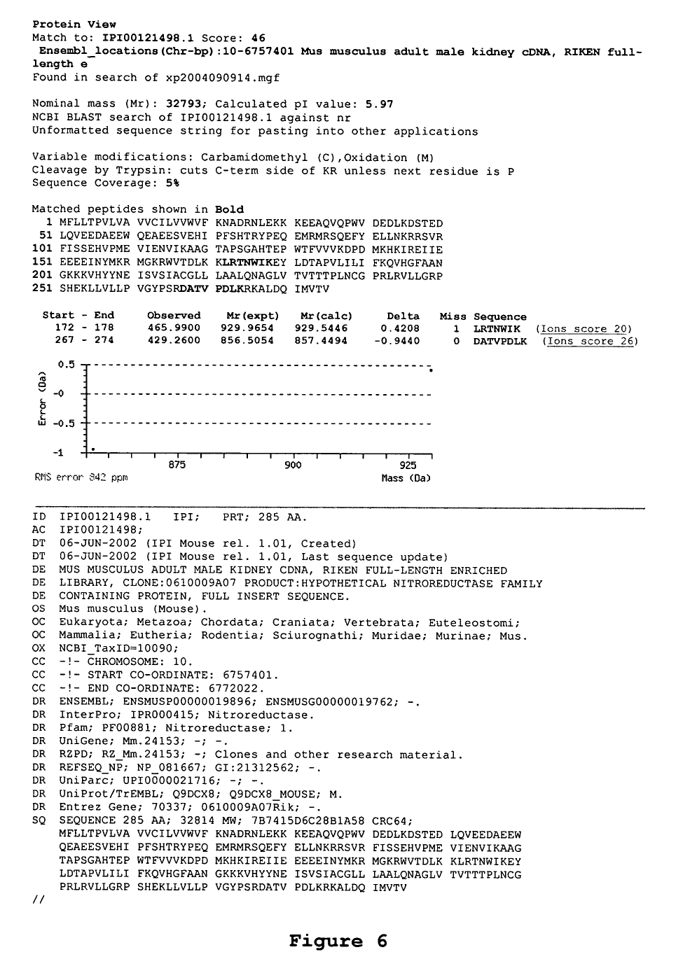

The isolated protein factor(s) that supports the growth of human embryonic stem cells has been further characterized in this Example. The one or more isolated factors from Example 1 were subjected to LC/MS/MS analysis using nano-spray LC/MS/MS mass spectrometer at the Michigan State University proteomics facility. FIG. 6 illustrates the protein view of the Mascot® search engine results (Matrix Science Inc., Boston, Mass.) of a factor from Example 1 subjected to the LC/MS/MS analysis. This analysis showed that the isolated factor was previously unknown and matched with a protein product as deduced from the cDNA sequence and identified using BlastP. According to the NCBI data the mouse cDNA (1541 bases, SEQ ID NO:2) encoded a deduced nitroreductase family containing protein with 285 amino acids (SEQ ID NO:1) and a mass of approximately thirty-three (33) kDa. The genomic DNA encoding the cDNA is 14,599 bps (SEQ ID NO:3) and maps on mouse chromosome 10 as shown in FIG. 6. It appeared to have homologs in rat and human which map on chromosomes 1 and 6, respectively. The human and rat homologs of this mouse gene have recently been shown to encode for iodotyrosine dehalogenase (Gnidehou et al. 2004. FASEB J. 18:1574-6; Solís-S et al. 2004. J. Endocrinol. 181: 385-392). Some homologous proteins to the mouse iodotyrosine dehalogenase include rat (SEQ ID NO: 7), human (SEQ ID NOs: 8, 9, 10), chimpanzee (SEQ ID NO: 11), orangutan (SEQ ID NO: 12) and pig (SEQ ID NO: 13) proteins.

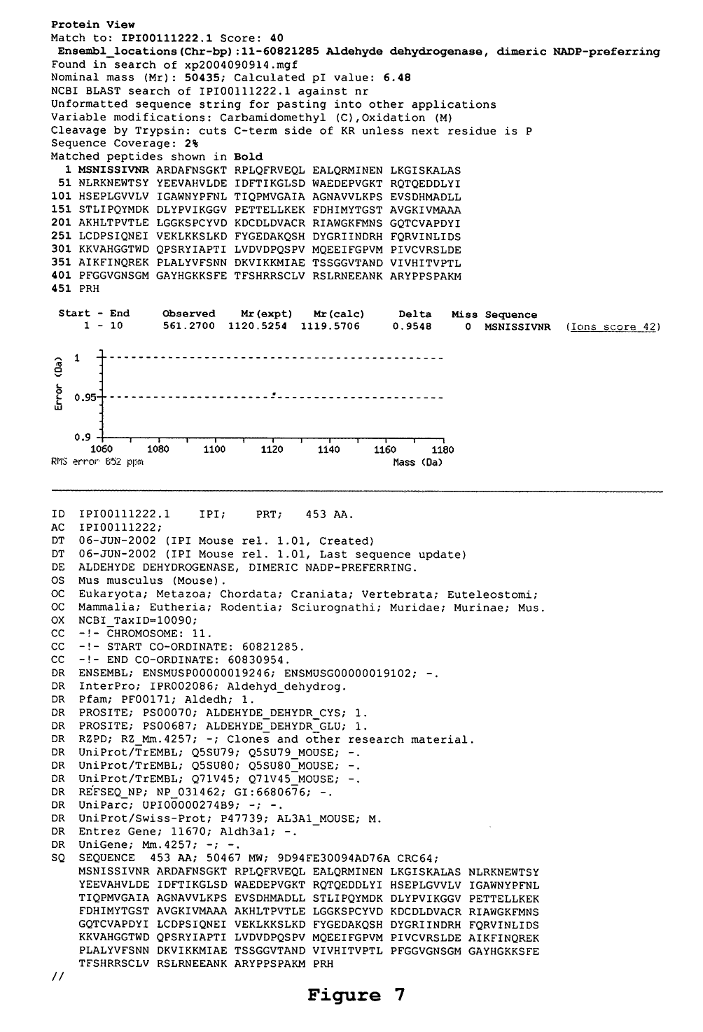

Proteomic analysis also identified a minor component in the sample supporting undifferentiated hES cell growth as a product of 453 amino acids (SEQ ID NO:4) encoded by an aldehyde dehydrogenase gene as shown in FIG. 7. FIG. 7 illustrates the protein view of the Mascot® search engine results (Matrix Science Inc., Boston, Mass.) of the minor factor after being subjected to the LC/MS/MS analysis using the nano-spray LC/MS/MS mass spectrometer. The size of the cDNA of the minor component is 1722 bases (SEQ ID NO:5) and the size of the genomic DNA is 9670 bps (SEQ ID NO:6). This aldehyde dehydrogenase gene and its product have been extensively investigated (Muzio et al. 2003. Chem Biol Interact. 143-144:37-43; Canuto et al. 2003. Chem Biol Interact. 143-144:29-35; Yin et al. 1989. Biochem Genet. 27:321-31; Santisteban et al. 1985. Ann Hum Genet. 49:87-100). However, its involvement in supporting hES cell growth has not been reported previously. Some homologous proteins to the mouse aldehyde dehydrogenase include other mouse (SEQ ID NOs: 14, 15, 16), rat (SEQ ID NOs: 17, 18), human (SEQ ID NOs: 19-22), dog (SEQ ID NO: 23) and cattle (SEQ ID NO: 24) proteins.

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the Claims attached herein.

I claim:

1. A medium for culturing stem cells while maintaining the stem cells in an undifferentiated state comprising one or more polypeptides homologous to iodotyrosine dehalogenase or aldehyde dehydrogenase.

2. The medium of claim 1 wherein the one or more polypeptides are recombinant.

3. A method of culturing stem cells while maintaining the stem cells in an undifferentiated state comprising culturing the stem cells in a medium comprising one or more polypeptides homologous to iodotyrosine dehalogenase or aldehyde dehydrogenase.

4. The method of claim 3 wherein the stem cells are human stem cells.

5. The method of claim 4 wherein the human stem cells are human embryonic stem cells.

6. The method of claim 3 wherein the one or more polypeptides are recombinant.

7. A method of culturing stem cells while maintaining the stem cells in an undifferentiated state comprising culturing the stem cells in a medium comprising one or more polypeptides having an amino acid sequence essentially identical to the amino acid sequences set forth in SEQ ID NO: 1 or SEQ ID NO: 4.

8. The method of claim 7 wherein the stem cells are human stem cells.

9. The method of claim 8 wherein the human stem cells are human embryonic stem cells.

10. The method of claim 7 wherein the one or more polypeptides are recombinant.

11. A method of preparing a partially purified conditioned medium for culturing stem cells while maintaining the cells in an undifferentiated state comprising:
(a) providing a medium;
(b) conditioning the medium by culturing fibroblasts in the medium;
(c) precipitating the conditioned medium with a first salt solution to provide a first set of fractions;
(d) selecting a fraction of the first set of fractions of the precipitated conditioned medium;
(e) applying the selected fraction to a cation-exchange resin;
(f) eluting a second set of fractions with a second salt solution; and
(g) selecting a fraction of the second set of fractions as the partially purified conditioned medium for culturing stem cells.

12. The method of claim 11 wherein the fibroblast are mouse embryonic fibroblasts.

13. The method of claim 11 wherein the second salt solution is a 500 mM potassium chloride solution.

14. A partially purified conditioned medium for culturing stem cells while maintaining the cells in an undifferentiated state prepared by the method comprising: providing a medium; conditioning the medium by culturing fibroblasts in the medium; precipitating the conditioned medium with a first salt solution to provide a first set of fractions; selecting a fraction of the first set of fractions of the precipitated conditioned medium; applying the selected fraction to a cation-exchange resin; eluting a second set of fractions with a second salt solution; and selecting a fraction of the second set of fractions as the partially purified conditioned medium for culturing stem cells.

15. The method of claim 14 wherein the fibroblast are mouse embryonic fibroblasts.

16. The method of claim 14 wherein the second salt solution is a 500 mM potassium chloride solution.

17. A method of culturing stem cells while maintaining the cells in an undifferentiated state comprising:
(a) providing stem cells for culturing in an undifferentiated state;
(b) providing partially purified conditioned medium for culturing stem cells while maintaining the cells in an undifferentiated state prepared by the method comprising: providing a medium; conditioning the medium by culturing fibroblasts in the medium; precipitating the conditioned medium with a salt to provide a first set of fractions; selecting one fraction of the first set of fractions of the precipitated conditioned medium; applying the selected fraction to a cation-exchange resin; eluting a second set of fractions; and selecting one of the second set of fractions as the partially purified conditioned medium to a culture of stem cells; and
(c) culturing the stem cells in a medium comprising a fresh culture medium and the partially purified conditioned medium.
